(19) United States
(12) Patent Application Publication
(10) Pub. No.: US 2011/0042325 A1
(43) Pub. Date: Feb. 24, 2011

(54) POLYMERIC DELIVERY VEHICLE FOR NANOPARTICLES

(75) Inventors: Achintya N. Bezbaruah, West Fargo, ND (US); Bret J. Chisholm, West Fargo, ND (US); Sita Krajangpan, Fargo, ND (US)

Correspondence Address: MCKEE, VOORHEES & SEASE, P.L.C., 801 GRAND AVENUE, SUITE 3200, DES MOINES, IA, 50309-2721, US

(73) Assignee: NDSU RESEARCH FOUNDATION, FARGO, ND (US)

(21) Appl. No.: 12/921,316

(22) PCT Filed: Mar. 6, 2009
(86) PCT No.: PCT/US2009/036370

§ 371 Date: Nov. 8, 2010

Related U.S. Application Data
(60) Provisional application No. 61/034,808, filed on Mar. 7, 2008.
(60) Provisional application No. 61/153,861, filed on Feb. 19, 2009.

(51) Int. Cl. C02F 1/68; B01J 20/26; C12N 11/08

(52) U.S. Cl. 210/747; 502/402; 435/180

(57) ABSTRACT

Efficient, targeted delivery of polymer entrapped nanoparticles, nutrients and microorganisms is provided by amphiphilic polysiloxane graft copolymers (APGC) based metal nanoparticle delivery vehicles configured to increase colloidal stability, reduce oxidation by non-target compounds, and have affinity towards water/contaminant interfaces.

### CROSS-REFERENCE TO RELATED APPLICATIONS

This application claims priority under 35 U.S.C. §119 of provisional applications having Ser. Nos. 61/034,808 filed Mar. 7, 2008 and 61/153,861 filed Feb. 19, 2009, which applications are hereby incorporated by reference in their entirety.

### GOVERNMENT SUPPORT

This invention was made with government support under North Dakota State

University Project # FAR0011378, USGS Award # 06HQGR0104 awarded by the United States Geological Survey. The government has certain rights in the invention.

### FIELD OF THE INVENTION

The present invention relates to the efficient, targeted delivery of polymer entrapped nanoparticles, nutrients and microorganisms. More particularly, the present invention relates to a new amphiphilic polysiloxane graft copolymers (APGC) based metal nanoparticle delivery vehicle configured to increase colloidal stability, reduce oxidation by non-target compounds, and have affinity towards water/contaminant interfaces.

### BACKGROUND OF THE INVENTION

An increasing number of laboratory and field studies illustrate the potential of metal particles for degrading organic and inorganic species susceptible to reduction reactions [1-5]. In the last decade, emphasis on metal particle use has changed from regular filings/particles to metal and bimetal nanoparticles [6-10]. Nanoparticles are attractive for remediation of various contaminants because of their unique physiochemical properties [11-13]. Various chlorinated aliphatic hydrocarbons [14-16] and toxic metals [15, 17-18] can be successfully remediated using metal nanoparticles such as zero valent iron (Fe^{o}) nanoparticles. Metal particles/nanoparticles have also been used for the remediation of ground water contaminated with chemicals used in explosives [19-22].

The effectiveness of a remediation approach depends on various factors, one of which is the ability to access the contaminant(s) with the metal nanoparticles [23-24]. Fe^{o} nanoparticles, for example, are highly reactive and react rapidly with surrounding media in the subsurface (dissolved oxygen and/or water). Thus, significant loss of reactivity occurs [25] before the particles are able to reach the target contaminant. To overcome this problem of oxidation, the in situ application of Fe^{o} particles is preceded by injection of a carbon source (e.g., liquid molasses) into the application site. The injection of a carbon source to render the site anoxic involves a major financial investment and is time consuming. Therefore, a need has been identified in the art to develop effective and efficient methods and systems to protect the metal nanoparticles from oxidation prior to their contact with the target contaminant.

Another important factor involved in the use of metal nanoparticles for remediation is their ability to individually disperse and suspend in water [26]. Fe^{o} nanoparticles tend to flocculate when added to water due to interparticle van der Waal interactions. Flocculation reduces the effective surface area of the metal and causes precipitation of the metal from the aqueous phase. Therefore, a further need has been identified in the art for systems and methods to form stable colloidal suspensions of metal nanoparticles in water.

In addition to protecting the surface of the metal nanoparticles from oxidation prior to contact with the contaminant forming stable colloidal suspensions in water, a further need in the art has been identified to create methods and systems providing an affinity between the metal nanoparticles and the water/contaminant interface. Maximum efficiency of the remediation approach will only be realized if the metal nanoparticles effectively migrate to the contaminant or the water/contaminant interface.

Therefore, it is a primary object, feature or advantage of the present invention to improve over the state of the art.

It is a further object, feature or advantage of the present invention to provide a polymeric delivery vehicle for nanoparticles that provides colloidal stability in water.

Another object, feature or advantage of the present invention is to provide a polymeric delivery vehicle for nanoparticles that protects the nanoparticles from oxidation by water and dissolved oxygen.

A still further object, feature or advantage of the present invention is to provide a polymeric delivery vehicle for nanoparticles that creates an affinity of the nanoparticles for the water/contaminant interface.

Yet another object, feature or advantage of the present invention is to provide a polymeric delivery vehicle that preserves the unique physiochemical properties of the nanoparticles, such as surface chemistry of the particles, to prevent agglomeration of the particles into clusters or flocks resulting in a loss of the effected surface area.

Another object, feature or advantage of the present invention is to provide a polymeric delivery vehicle for nanoparticles exhibiting a hydrophobicity to water to protect the nanoparticles from oxidation by water and thereby creating a barrier to water while also creating an affinity of the nanoparticles for the water/contaminant interface.

A still further object, feature or advantage of the present invention is to provide a polymeric delivery vehicle for nanoparticles having functionalized amphiphilic graft copolymers for delivering nanoparticles to the water/contaminant interface.

Yet another object, feature or advantage of the present invention is to provide a polymeric delivery vehicle for nanoparticles having an anchoring group, such as carboxylic acid or alkoxysilane groups, in conjunction with a polymer backbone for efficient absorption of polymer molecules onto the surface of the nanoparticles while the water-soluble grafts, such as poly(ethylene glycol), allows for dispersibility and colloidal stability in water.

These and/or other objects, features or advantages of the present invention will become apparent from the following specification and claims that follow. No single embodiment of the present invention need achieve all or any particular number of the foregoing object, feature, or advantage of the present invention.

### SUMMARY OF THE INVENTION

According to one aspect of the present invention, a targeted remediation agent delivery system is disclosed. The system includes a remediation agent having a contaminant neutralizing property for remediating ground water, and a copolymer adapted to entrap the remediation agent to protect from reactivity with surrounding media while in transport to and while at a remediation site to ensure the remediation agent accesses and neutralizes the contaminant. In a preferred form, the system also includes the remediation agent being an iron nanoparticle, the polymer being a functionalized amphiphilic graft copolymer, the amphiphilic graft copolymer includes a polysiloxane polymer backbone to facilitate permeation of the contaminant onto a surface of the iron nanoparticle, the functionalized amphiphilic graft copolymer includes a water soluble graft to facilitate dispersibility and colloidal stability in water. The water soluble graft includes polyethylene glycol, the functionalized amphiphilic graft copolymer includes an anchoring group, and the anchoring group includes carboxylic acid or an alkoxysilane group.

According to another aspect of the present invention, a new method for targeted delivery of a remediation agent to a remediation site for the remediation of a ground water contaminant is disclosed. The method includes providing a copolymer transport, entrapping the remediation agent within the copolymer transport, transporting the remediation agent with the copolymer transport to the remediation site, and keeping the remediation agent from reacting with surrounding media while in transport to and while at the remediation site to access and neutralize the ground water contaminant. In a preferred form, the method also includes the remediation agent being an iron nanoparticle wherein the copolymer is a functionalized amphiphilic graft copolymer having a polysiloxane copolymer backbone. The method also includes the step of permeating the contaminant onto a surface of the iron nanoparticle, adding a water soluble graft of polyethylene glycol to the functionalized amphiphilic graft copolymer, controlling dispersibility and colloidal stability of the functionalized amphiphilic graft copolymer in water using the water soluble graft, and adding an anchoring group of carboxylic acid or an alkoxysilane group.

### BRIEF DESCRIPTION OF THE FIGURES

FIG. 1 is a schematic representation of synthesized amphiphilic polysiloxane graft copolymers (APGCs).

FIG. 2 is a schematic of the hydrosilylation between hydride-functional polysiloxane and mono-functional vinyl compounds.

FIG. 3 is a schematic representation of the polymer coated nanoparticles both in water and at the water/contaminant interface.

FIG. 4 is a TEM image of synthesized nZVIhaving a magnified portion shown in the inset image.

FIG. 5 is an XRD spectrum of synthesized iron nanoparticles.

FIG. 6 is an SEM image and EDX spectrum of synthesized nZVI.

FIG. 7 is a schematic of the APGC synthesis process according to an exemplary embodiment of the present invention.

FIG. 8 is an H NMR spectrum of APGC.

FIG. 9 is a plot of sedimentation studies for APGC coated nZVI.

FIG. 10 is a plot of dechlorination curves for bare nZVI and APGC coated nZVI.

FIG. 11a-c are microscopic images of organic compounds (e.g., TCE, APGC and TCE+APGC) and a polymer in water.

FIG. 12 is a plot of sedimentation studies for characterizing “shelf-life” for APGC coated nZVI.

FIG. 13 is a plot of sedimentation studies for APGC coated nZVI in water having varying ionic strengths.

### DETAILED DESCRIPTION

The present invention is directed towards systems and methods for a polymeric delivery vehicle for nanoparticles.

Much of the interest in nanoparticles stems from their extremely high surface area. While many procedures for the synthesis of a wide array of nanoparticles have been developed, delivery of the nanoparticles in a nonaggolmerated form to a specific region where they are needed remains a significant area of interest.

For groundwater remediation using Fe^{o} nanoparticles, effectiveness will depend on the ability to deliver the Fe^{o} nanoparticles to the water/contaminant interface without flocculation and severe oxidation. To accomplish effective delivery of Fe^{o} nanoparticles, a delivery system that provides for dispersibility and colloidal stability of individual Fe^{o} nanoparticles in water is required. In addition, the delivery system should protect the Fe^{o} nanoparticles from severe oxidation by water and provide an affinity for the water/contaminant interface. Although Fe^{o} nanoparticles are addressed herein, it should be appreciated by those skilled in the art that the present invention is not limited to Fe^{o} nanoparticle delivery. For example, the systems and methods of the present invention could be used to transport other nanoparticle types as well as nutrients and microorganisms to a water/contaminant interface.

Colloidal stability of Fe^{o} nanoparticles has been accomplished using surfactants [27]. The hydrophobic “tails” of the surfactants physically absorb on the Fe^{o} nanoparticle surface while the hydrophilic “heads” inhibit flocculation and allow for suspension in the aqueous medium. While surfactants enable colloidal stability in water, the highly reversible nature of surfactant absorption limits application as a delivery system for ground water decontamination since desorption will be favored when the nanoparticles are transported through surfactant-free ground water. In contrast, high molecular weight, amphiphilic polymers show essentially irreversible absorption and, thus, are more suitable as a delivery system for ground water remediation [28-29].

Saleh, et al. [30] have shown that amphiphilic triblock copolymers with an A-B-C triblock microstructure are effective delivery systems for Fe^{o} nanoparticles. The chemical composition of the A, B, and C blocks were poly(methacrylic acid), poly(methyl methacrylate), and poly(styrenesulfonic acid), respectively. The triblock copolymers were produced using atom transfer radical polymerization (ATRP) in conjunction with a post-polymerization ester-hydrolysis step and a post-polymerization sulfonation step. Since it was previously known that carboxylic acid groups absorb strongly on to iron oxide surfaces, the purpose of the poly(methacrylic acid) block was to absorb or anchor the polymer molecules to nanoparticle surfaces while the hydrophilic, polyanion block, poly(styrene sulfonic acid), was utilized to provide colloidal stability. The hydrophobic poly(methyl methacrylate) block was expected to reduce excessive oxidation of the Fe^{o} nanoparticles by water and enhance the affinity of the nanoparticles for the water/contaminant interface. The results of the research demonstrated enhanced colloidal stability and an increased affinity for a water/organic interface provided by the amphiphilic triblock copolymer.

While the amphiphilic triblock copolymers synthesized and evaluated by Saleh, et al. [30] showed promising results with respect to enhanced colloidal stability of Fe^{o} nanoparticles and the creation of a thermodynamic affinity of the nanoparticles for the water/contaminant interface, kinetic studies showed a decrease in the rate of contaminant degradation for the polymer-modified Fe^{o} nanoparticles as compared to unmodified Fe^{o} nanoparticles [31]. The reduction in contaminant degradation rate was attributed to low permeability of the contaminant through the poly(methyl methacrylate) film absorbed on the Fe^{o} nanoparticles. In addition to issues associated with contaminant degradation rate, the synthesis method required to produce the triblock copolymer structure is quite sensitive to impurities and oxygen and quite slow. Further, catalyst residues can be difficult to remove. Despite 15 years of intense research, ATRP is not being practiced commercially to any great extent. As a result, there exists a need to prepare tailored, multifunctional polymeric materials using a cost-effective, commercially-viable synthetic route that could serve as a highly effective delivery system for Fe^{o} nanoparticles.

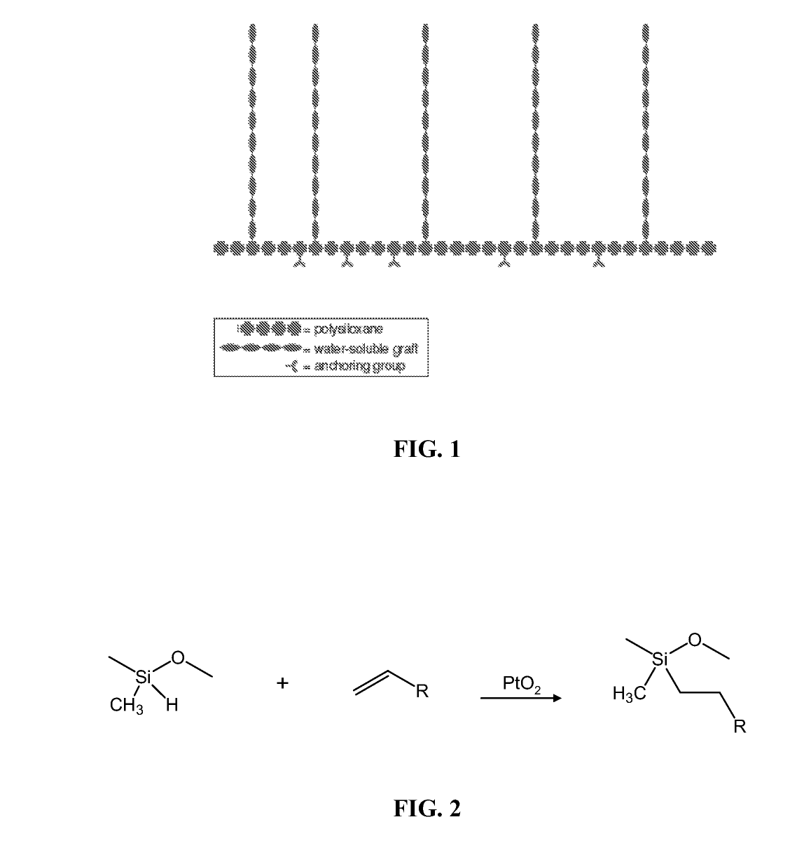

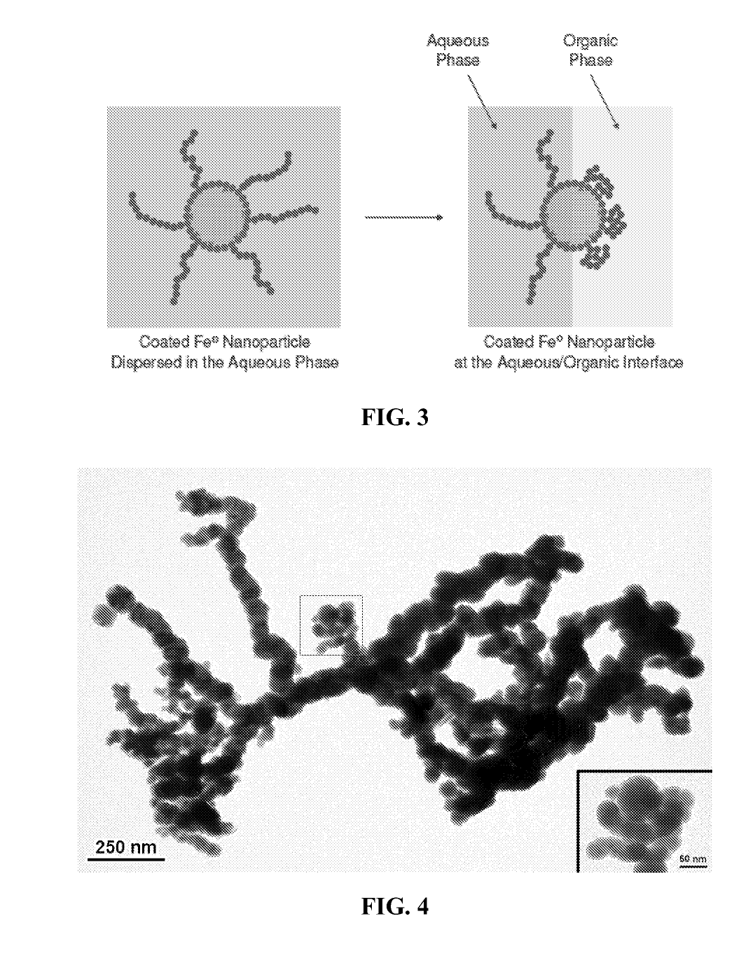

Considering the requirements of an effective delivery vehicle for nZVI (e.g., Fe^{o} nanoparticles) functionalized amphiphilic polysiloxanes are an ideal class of polymers for the application. Amphiphilic polysiloxane graft copolymers (APGCs) were synthesized in laboratory for this purpose as shown in FIG. 1. The synthesis involved hydrosilylation between hydride-functional polysiloxanes and monofunctional vinyl compounds (see FIG. 2). The carboxylic acid anchoring group in conjunction with the polysiloxane polymer backbone allows for efficient absorption of polymer molecules onto the surface of the nZVI while the water-soluble grafts, such as poly ethylene glycol (PEG), allow for dispersibility and colloidal stability in an aqueous medium. Polysiloxane used to coat the surface of nZVI is hydrophobic in nature. Because of hydrophobicity the polysiloxane polymer will have a high affinity to exist at the water/contaminant interface as shown in FIG. 3. The polymer being non-polar will also readily allow permeation of non-polar contaminants to the nZVI surface.

nZVI Synthesis

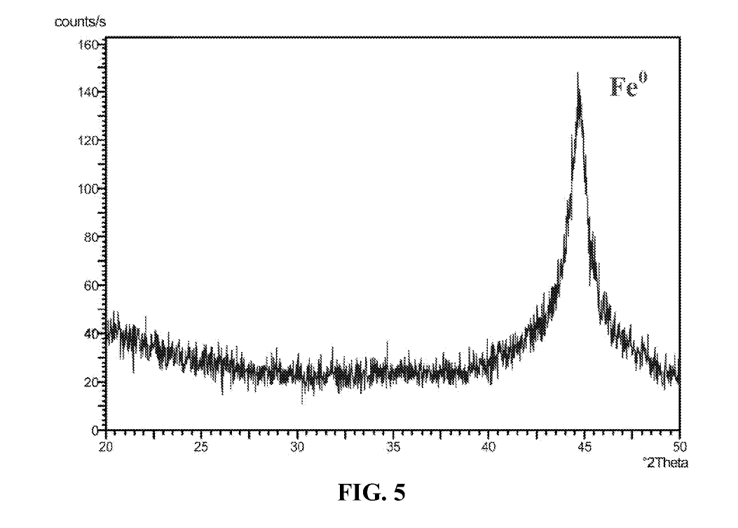

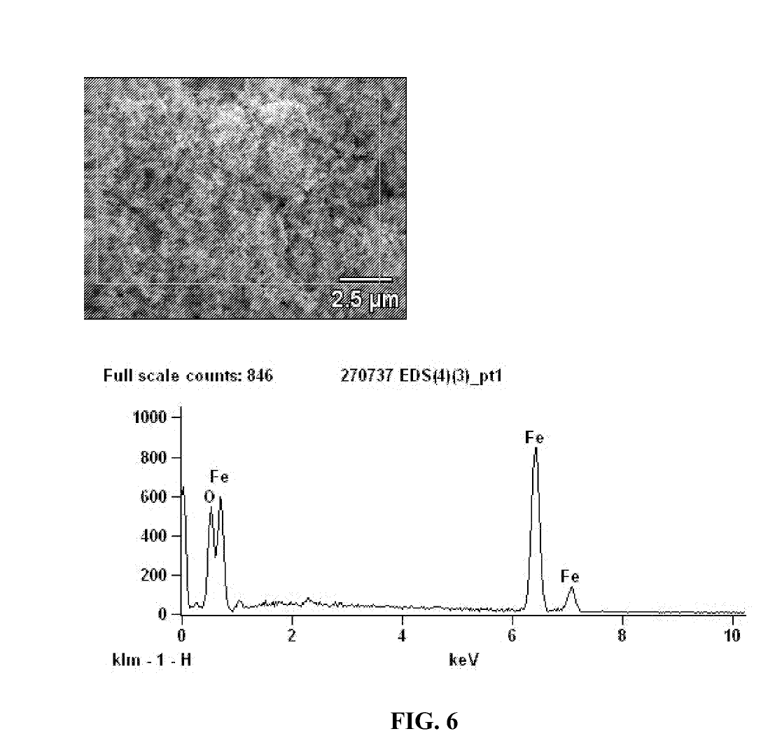

The nZVI may be synthesized by borohydride reduction of ferrous ion in FeSO₄.7H₂O (Aldrich, USA) in an aqueous phase. The synthesized particles have shown both high reactivity and durability. The synthesized nanoparticles are dried under alternating N₂ and vacuum overnight followed by overnight air stabilization to passivate the iron. The nZVI may be stored in a nitrogen environment in a glovebox (Innovative Technology, USA) for later use. The synthesized nZVI are then characterized using TEM. The particle size of nZVI synthesized varied from 10 to <100 nm with an average diameter of ˜35 nm (see FIG. 4). The BET specific surface was determined to be 25 m²/g. X-ray diffraction (XRD) analysis of nZVI may also be performed on a Philips X′Pert MPD with Cu K_X-ray source. Analysis can be performed at 40 kV and 30 mA with a scan range from 20° to 80°. XRD spectrum showed that only Fe⁰ in the synthesized nZVI (see FIG. 5). SEM/EDS data indicates that iron is the most abandon mineral (84.34%), with a smaller amount of oxygen (15.66%, in the oxide shell) on the nZVI (see FIG. 6). An oxide shell (˜2.5 nm) of amorphous FeOOH is clearly visible around the nanoparticles (see inset image in FIG. 4). The shell prevents particles from spontaneously igniting in the atmosphere, yet allowing contaminant access to nZVI in solution.

### APGCs Synthesis

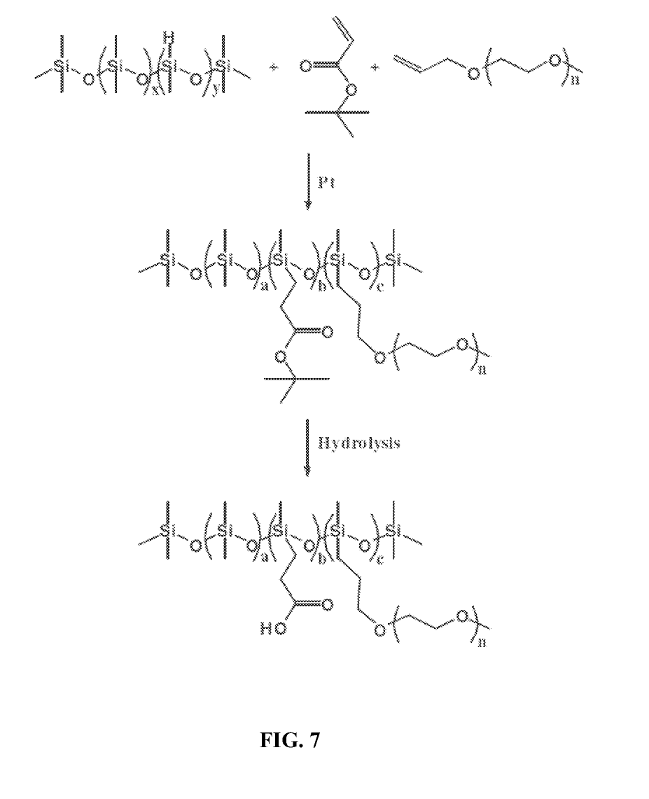

APGCs may be synthesized by hydrosilylation under N₂ atmosphere at 90° C. PtO₂ may be used as the catalyst for hydrosilylation. The synthesis from hydride-functional polysiloxanes and monofunctional vinyl compounds, both commercially available, is shown in FIG. 7.

A PDMS-g-PEG graft copolymer containing pendant carboxylic acid groups may be synthesized by dissolving 20.4 mmol hydride of a poly(methylhydrosiloxane-dimethylsiloxane) copolymer (PDMS) (MW 2000 g, EW 490 g (20.4 mmol hydride), HMS-151, Gelest), 10.2 mmol of monoallyl-functional polyethyleneglycol (PEG, MW 350) (3.57 g, 10.2 mmol, Clariant, USA), and 10.2 mmol of tert-butylacrylate (tBA) (1.57 g, 10.2 mmol, Aldrich, USA) in toluene (Aldrich). A catalytic amount of PtO₂ (Aldrich, USA) may be added, and the mixture heated at 90° C. overnight. Upon completion of the reaction, the reaction mixture is cooled to room temperature (25±2° C.). Platinum oxide may be removed by vacuum filtration, and the polymer (PDMS/PEG/tBA) isolated by vacuum stripping the toluene.

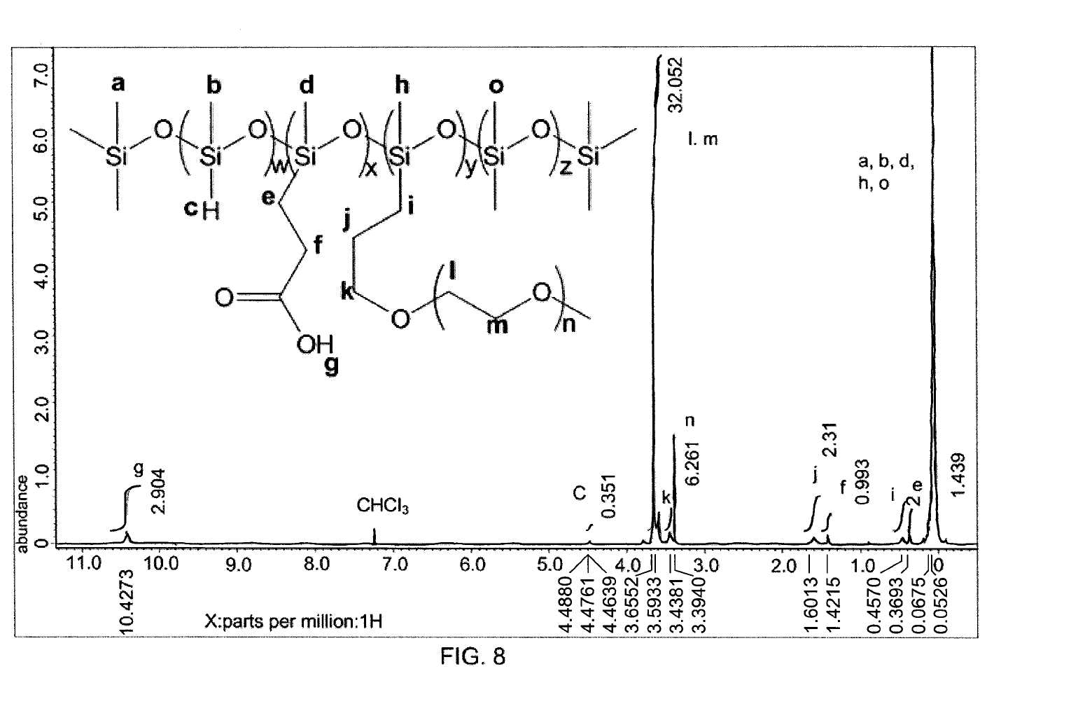

To generate the carboxylic acid anchoring groups, the tert-butyl ester groups of the graft copolymer are hydrolyzed as follows. PDMS/PEG/tBA (10 g) was dissolved in 25 ml of dichloromethane. Trifluoroacetic acid (TFA) (Aldrich) is added to the solution (5 mol equivalent), and the mixture is stirred at room temperature overnight to complete the reaction. The carboxylic acid-functional graft copolymer may be isolated by vacuum stripping the dichloromethane, TFA, and tert-butanol. Each synthesis step can be monitored using H-NMR as shown in FIG. 8. The analyzed C-NMR, and FTIR spectra are illustrated in FIG. 8.

Carboxylic acid-functional APGC containing polyethylene glycol grafts (PDMS/PEG/AA) are successfully synthesized, as shown. According to one aspect of the present invention, APGCs are synthesized by hydrosilylation using PtO₂ as a catalyst. The proton absorption peaks at δ 0.4-0.5 ppm and δ 1.4-1.6 ppm in the H NMR spectrum correspond to methylene protons created as a result of successful hydrosilylation of the vinyl functional precursors to the hydride functional polysiloxane copolymer.

The carbon absorption C NMR spectrum (Model JEOL ECA 400 MHz, NMR Spercrometer) verified that the hydrosilylation reaction had occurred. The carbonyl peak has a quartet at δ 158.0-158.9 ppm. Poly(ethylene glycol) peaks appear between δ 59 ppm and δ 78 ppm. The tert-butyl carbonyl and its methylene carbon are located at δ 150.6 ppm, and δ 29.0 ppm, respectively. The same procedure was used to produce APGC varying the relative molar concentration of carboxylic acid groups to PEG grafts.

With the varied mass ratios, five formulations of PDMS/PEG/AA, A (70/25/5), B (62/36/2), C (72.5/21/6.5), D (67/29/4), and E (65/32/3), may be synthesized and characterized.

The FT-IR spectroscopy (Model: Vertex 70, Bruker) technique may also be used to examine the polymers before (PDMS/PEG/tBA) and after hydrolysis (PDMS/PEG/AA) to make sure that the reaction was complete. After hydrolysis the peak at 1392 cm⁻¹ and the sidearm to the peak at 1408 cm⁻¹ disappeared indicating transformation of tert butyl acrylate via hydrolysis reaction. Correspondingly, FT-IR spectra showed a noticeable change in the carbonyl peak in both size and position (1681 cm⁻¹ and 1786 cm⁻¹ before and after hydrolysis, respectively). The band of peaks between 2800 and 3000 cm⁻¹ became slightly broader after hydrolysis and another peak at 1170 cm⁻¹ appeared because of the carbonyl peak of acrylic acid.

Coated nZVI and Colloidal Stability

According to one aspect of the present invention, coated nZVI and colloidal stability studies included the combination of nZVI (60 mg) with 20 ml of PDMS/PEG/AA emulsion at various concentrations (5 gL⁻¹, 10 gL⁻¹, and 15 gL⁻¹) of the polymer. The mixtures may be sonicated for 30 min to prevent flocculation of nZVI and mixed in a custom made end-over-end rotator (28 rpm) for 72 hours to allow the graft PDMS/PEG/AA to adsorb onto the surface of nZVI. The APGC coated nZVI may then be centrifuged (1800 rpm, Model Heraeus Labofuge 400R Centrifuge, Thermo Electron Corporation, USA) and washed multiple times to remove any excess non-adsorbed APGC. Evaluation of colloidal stability of the coated nZVI may by measured by observing sedimentation rates of the coated nZVI suspension using UV spectrophotometer (Model Cary 50000, 508 nm, Varian, USA). The sedimentations of the PDMS/PEG/AA coated nZVI are observed for a period of time (e.g., 2 hours). The same evaluation was performed for the control using 60 mg of uncoated/bare nZVI in 20 mL deionized water. A 2 hour experimentation time is considered sufficient as nZVI reacts very fast with contaminants and provides complete utilization of the iron, which means good dispersion and colloidal stability are achieved and should be sufficient in remediation applications.

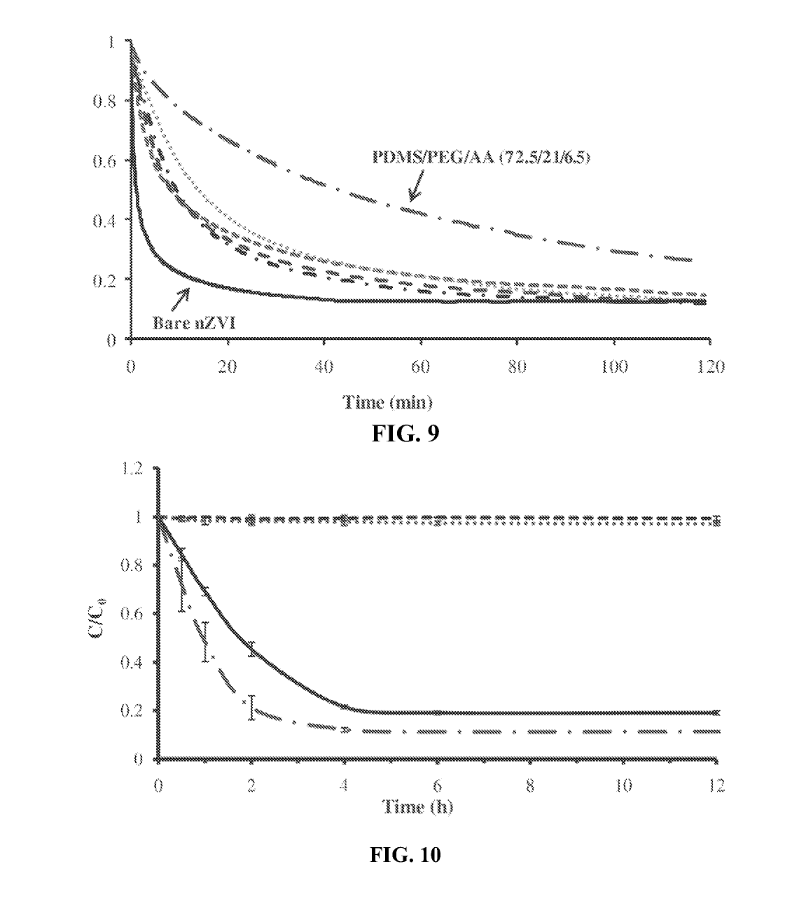

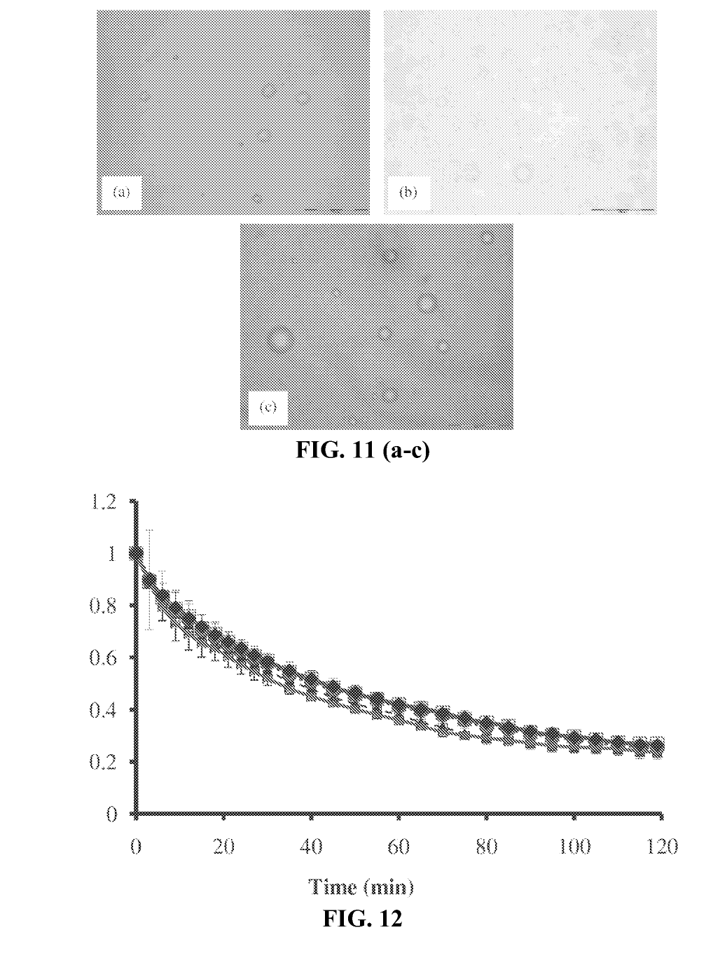

Three concentrations (5, 10, 15 gL⁻¹) of PDMS/PEG/AA (APGC) may be used to coat nZVI for conducting sedimentation studies. The nZVI coated with 15 gL⁻¹ APGC formed the most stable suspension as compared to lower APGC concentrations and bare nZVI. Further analyses conducted with the nZVI coated with 15 μL⁻¹ APGC, and particularly, the impact of changing PEG/AA ratio on sedimentation rate was studied for 15 gL⁻¹ APGC as shown in FIG. 9. FIG. 9 indicates that the APGC with the highest concentration of acrylic acid anchoring groups provided the highest colloidal stability. Specifically, FIG. 9 shows sedimentation studies of APGC coated nZVI. In FIG. 9, PDMS is Polydimethylsiloxane, PEG is polyethylene glycol, and AA is acrylic acid. Further, the ratio within parentheses represents weight percent of each component. For example, for components PDMS, PEG and AA, exemplary weight percentages include, but are not limited to, 70/25/5, 62/36/2, 72.5/21/6.5, 67/29/4, and 65/32/3. Notably, marked decrease in sedimentation rate was observed for APGC modified nZVI as compared to bare nZVI. The following legend provides added description for the various weight percent and components graphed in FIG. 9. For components PDMS/PEG/AA: the component weight percent of 70/25/5 is indicated by a dash-dot line, the component weight percent of 62/36/2 is indicated by a dash-dash line, the component weight percent 72.5/21/6.5 is indicated by a long dash-dot line, the component weight percent 67/29/4 is indicated by a dotted line, and the component weight percent 65/32/3 is indicated by bolded dashed line. The bare nZVI is indicated in FIG. 9 by a solid line.

### Degradation

According to one aspect of the present invention, batch experiments were conducted in 20 ml amber glass bottles with Teflon septa. Trichloroethylene (TCE, Aldrich, USA) was used as test contaminants. The initial TCE concentration was 30 mg L⁻¹. Bare nZVI (1 gL⁻¹) and APGC coated nanoparticles (1 gL⁻¹ of nZVI) were used. Controls with TCE solution and only APGC were run simultaneously. Blanks were run with only TCE solution. Aliquots were withdrawn from sacrificial batch reactors at definite intervals and analyzed using a GC/MS (Model 5975, Agilent, USA). The TCE dechlorination curves by bare nZVI and APGC coated nZVI along with control and blank are shown in FIG. 10. In another aspect, initial concentrations of TCE (30 mg L⁻¹) and nZVI (1 g L⁻¹) are used. The APGC coated nZVI exhibits greater degradation rates for TCE with over 90% removal within approximately 5 hours. No significant decrease of TCE was observed either in the control or the blank. Two-way analysis of variance test on the degradation data indicates that there is a significant difference between TCE degradation by bare nZVI and APGC coated nZVI (α=0.05>ρ-value=0.003) and coated nZVI performed better (6% degradation). The statistical test also indicated that there is no significant difference between the blank and control (α=0.05<ρ-value=0.135).

To evaluate the affinity of the synthesized APGC microscopic observations were made. A droplet size of (a) TCE solution, (b) water when only APGC (no TCE) was present and (c) TCE solution when APGC was present were observed using an optical microscope attached with a digital camera. The size of the droplets may be measured using image analysis software. The average droplet diameters of only TCE solution (no APGC present) and only water in the presence of APGC were ˜18.2 mm and 3 μm, respectively. In the presence of APGC, the droplet diameter of TCE solution was ˜24.7 μm which is larger than the earlier two cases. The larger droplet size is an indicator of the affinity of APGC for organic contaminants.

### Shelf-Life

The polymer coated nanoparticles need to have long shelf-life to be commercially viable. Colloidal stability of APGC coated nZVI as relating to shelf-life was subsequently investigated. Many batches of APGC coated nZVI were prepared and stored in a cabinet at room temperature (25±2° C.). Sedimentation studies were conducted for multiple batches (in triplicates) every month. The result showed that the colloidal stability of coated nZVI remained unchanged after 5.5 months (see FIG. 11). A 1 month shelf-life is indicated by the line with diamond shapes, a 2 month shelf-life is indicated by the line with square shapes, a 3 month shelf-life is indicated by the line with triangle shapes, a 4 month shelf-life is indicated by the line with circles, and a 5.5 month shelf-life is indicated by the line with asterisks.

### Effect of Ionic Strength on Colloidal Stability

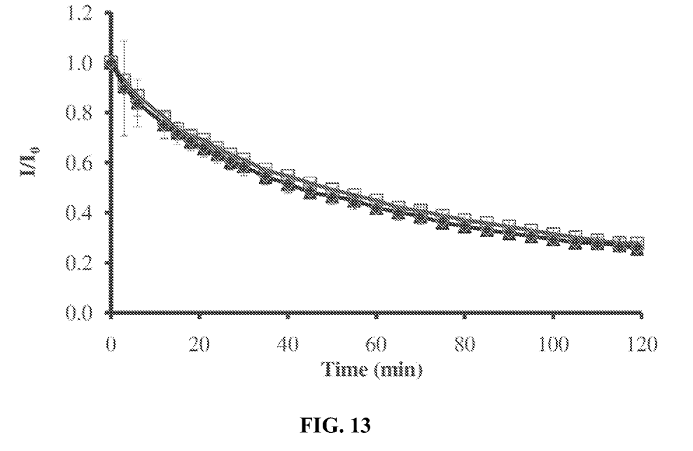

Sedimentation studies for APGC coated nZVI were conducted in salt solutions to observe the effect of ionic strength. The sodium chloride (NaCl) concentrations prepared included 0 mM, 5 mM, and 10 mM solutions. Three replicates were conducted for each NaCl concentration. The results showed that there was no significant difference in sedimentation in 0 mM (represented by line with diamond shapes), 5 mM (represented by line with triangle shapes), 10 mM (represented by line with square shapes) NaCl solutions (see FIG. 13).

The present invention provides for synthesis of APGCs. According to one aspect, the synthesis process includes, but is not limited to, the hydrosilylation of tert-butylacrylate and monoallyl-functional PEG to a polysiloxane copolymer containing hydride groups and the subsequent hydrolysis of the tertbutylester groups. In another aspect of the present invention, nZVI was treated with APGCs. The treatment enhances nanoparticle colloidal stability in water and the magnitude of the enhancement was found to be a function of APGC chemical composition. For example, the APGC possessing the highest concentration of carboxylic acid anchoring groups provided the highest colloidal stability. Further, the reduction rate of TCE by APGCs coated nZVI is greater as compared to bare nZVI. The APGC coated nZVI remains reactive and is effective in reducing TCE due to greater colloidal stability providing more reactive surface area.

The embodiment of the present invention have been set forth in the drawings and specification and although specific terms are employed, these are used in a generically descriptive sense only and are not used for the purposes of limitation. Changes in the formed proportion of parts, as well as in the substitution of equivalences are contemplated as circumstances may suggest or are rendered expedient without departing from the spirit and scope of the invention as further defined in the following claims.

### REFERENCES

All references listed throughout the specification, including the references listed below are herein incorporated by reference in their entireties.
1. Fennelly, J. P.; Roberts, A. L. Reaction of 1,1,1-Trichloroethane with Zero-Valent Metals and Bimetallic Reductants, Environ. Sci. Technol., 32:1980-1988, 1998.
2. Arnold, W. A.; Roberts, A. L. Pathways of Chlorinated Ethylene and Chlorinated Acetylene Reaction with Zn(0), Environ. Sci. Technol., 32:3017-3025, 1998.
3. Kim, Y.-H.; Carraway, E. R. Dechlorination of Pentachlorophenol by Zero Valent Iron and Modified Zero Valent Irons, Environ. Sci. Technol., 34:2014-2017, 2000.
4. Xu, Y.; Zhang, W.-X. Subcolloidal Fe/Ag Particles for Reductive Dehalogenation of Chlorinated Benzenes, Ind. Eng. Chem. Res., 39: 2238-2244, 2000
5. Doong, R.; Chen, K.; Tsai, H. Reductive Dechlorination of Carbon Tetrachloride and Tetrachloroethylene by Zerovalent Silicon-Iron Reductants, Environ. Sci. Technol., 37: 2575-2581, 2003.
6. Wang, C.-B.; Zhang, W.-X. Synthesizing Nanoscale Iron Particles for Rapid and Complete Dechlorination of TCE and PCBs, Environ. Sci. Technol., 31:2154-2156, 1997.
7. Elliott, D. W.; Zhang, W.-X. Field Assessment of Nanoscale Bimetallic Particles for Groundwater Treatment, Environ. Sci. Technol., 35:4922-4926, 2001.
8. Tee, Y.-H.; Grulke, E.; Bhattacharyya, D. Role of Ni/Fe Nanoparticle Composition on the Degradation of Trichloroethylene from Water, Ind. Eng. Chem. Res., 44:7062-7070, 2005.
9. Liu, Y.; Majetich, S. A.; Tilton, R. D.; Sholl, D. S.; Lowry, G. V. TCE Dechlorination Rates, Pathways, and Efficiency of Nanoscale Iron Particles with Different Properties, Environ. Sci. Technol., 39:1338-1345, 2005.
10. Zhang, W.-X. Karn, B. Nanoscale Environmental Science and Technology: Challenges and Opportunities, Environ. Sci. Technol. A-Pages, 39:94A-95A, 2005.
11. Zhang, W.-X. Nanoscale Iron Particles for Environmental Remediation: An Overview, J. Nanoparticle Res., 5:323-332, 2003.
12. Li, X.-Q, Brown, D. G., Zhang, W.-X. Stabilization of Biosolids with Nanoscale Zero-valent Iron (nZVI), J. Nanoscale Res., 9:233-243, 2007.
13. Gillham, R. W.; O'Hannesin, S. F. Enhanced Degradation of Halogenate Aliphatics by Zero-valent Iron, Ground Water, 32:958-967, 1994.
14. Li, X.-Q., Elliott, D. W., Zhang, W.-X. Zero-valent Iron Nanoparticles for Abatement of Environmental Pollutants: Materials and Engineering Aspects, Cri. Rev. Solid State Mat. Sc., 31:111-122, 2006.
15. Liu, Y., Lowry, G. V. Effect of Particle Age (Feo Content) and Solution pH on NZVI Reactivity: H2 Evolution and TCE Dechlorination, Environ. Sc. Technol., 40:6085-6090, 2006.
16. Blowes, D. W.; Ptacek, C. J.; Jambor, J. L., In-Situ Remediation of Cr(VI) Contaminated Groundwater Using Permeable Reactive Walls: Laboratory Studies, Environ. Sci. Technol., 31:3348-3357, 1997.
17. Fiedor, J. N.; Bostick, W. D.; Jarabek, R. J.; Farrell, J. Understanding the Mechanism of Uranium Removal from Groundwater by Zero-Valent Iron Using X-ray Photoelectron Spectroscopy, Environ. Sci. Technol., 32:1466-1473, 1998.
18. Li, X.-Q., Zhang, W.-X. Sequestration of Metal Cations with Zerovalent Iron Nanoparticles-A Study with High Resolution X-ray Photoelectron Spectroscopy (HR-XPS), J. Phys. Chem., 111:6939-6946, 2007.
19. Gregory, K. B.; Larese-Casanova, P.; Parkin, G. F.; Scherer, M. M. Abiotic Transformation of Hexahydro-1,3,5-trinitro-1,3,5-triazine by Felt Bound to Magnetite, Environ. Sci. Technol., 38:1408-1414, 2004.
20. Hundal, L.,; Singh, J.; Bier, E. L.; Shea, P. J.; Comfort, S. D.; Powers, W. L. Removal of TNT and RDX from Water and Soil Using Iron Metal, Environmental Pollution, 97:55-64, 1997.
21. Singh, J.; Comfort, S. D.; Shea, P. J. Iron-Mediated Remediation of RDXContaminated Water and Soil under Controlled Eh/pH, Environ. Sci. Technol., 33:1488-1494, 1999.
22. Oh, S.-Y.; Chiu, P. C.; Kim, B. J.; Cha, D. K. Zero-valent Iron Pretreatment for Enhancing the Biodegradability of RDX, Water Res., 39:5027-5032, 2005.
23. Buffle, J.; Wilkinson, K. J.; Stoll, S.; Filella, M.; Zhang, J. A Generalized Description of Aquatic Colloidal Interactions: The Three-colloidal Component Approach, Environ. Sc. Technol., 32:2887-2899, 1998,
24. Schrick, B., Hydutsky, B. W.; Blough, J. L.; Mallouk, T. E. Delivery Vehicles for Zerovalent Metal Nanoparticles in Soil and Groundwater, Chem. Mater., 16:2187-2193, 2004.
25. He, F; Zhao, D. Preparation and Characterization of a New Class of Starch-Stabilized Bimetallic Nanoparticles for Degradation of Chlorinated Hydrocarbons in Water, Environ. Sc. Technol., 39:3314-3320, 2005.
26. Phenrat, T., Saleh, N., Sirk, K., Tilton, R. D., Lowry, G. V. Aggregation and Sedimentation of Aqueous Nanoscale Zerovalent Iron Dispersion, Environ. Sc. Technol., 41:284-290, 2007.
27. Rosen, M. J. “Surfactants and Interfacial Phenomena” 3rd ed.; Wiley-Interscience: New York, 2002.
28. Velegol, S. B.; Tilton, R. D. A Connection between Interfacial Self-Assembly and the Inhibition of Hexadecyltrimethylammonium Bromide Adsorption on Silica by Poly-L-lysine, Langmuir, 17:219-227, 2001.
29. Braem, A. D.; Biggs, S.; Prieve, D. C.; Tilton, R. D. Control of Persistent Nonequilibrium Adsorbed Polymer Layer Structure by Transient Exposure to Surfactants, Langmuir 19:2736-2744, 2003.
30. Saleh, N.; Phenrat, T.; Sirk, K.; Dufour, B.; Ok, J.; Sarbu, T.; Matyjaszewski, K.; Tilton, R. D.; Lowry, G. V. Adsorbed Triblock Copolymers Deliver Reactive Iron Nanoparticles to the Oil/Water Interface, Nano Lett., 5:2489-2494, 2005.
31. Saleh, N., Sirk, K., Liu, Y., Phenrat, T., Dufour, B., Matyjaszewski, K., Tilton, R. D., Lowry, G. V. Surface Modifications Enhance Nanoiron Transport and NANPL Targeting in Saturated Porous Media, Environ. Eng. Sc., 24:45-57, 2007.

What is claimed is:

1. A targeted remediation agent delivery system, comprising:
a remediation agent having a contaminant neutralizing property for remediating groundwater;
a copolymer adapted to entrap the remediation agent to protect from reactivity with surrounding media while in transport to and while at a remediation site to ensure the remediation agent accesses and neutralizes the contaminant.

2. The system of claim 1 wherein the remediation agent comprises an iron nanoparticle.

3. The system of claim 1 wherein the copolymer comprises a functionalized amphiphilic graft copolymer.

4. The system of claim 3 wherein the functionalized amphiphilic graft copolymer comprises a polysiloxane polymer backbone to facilitate permeation of the contaminant onto a surface of the iron nanoparticle.

5. The system of claim 3 wherein the functionalized amphiphilic graft copolymer comprises a water soluble graft to facilitate dispersibility and colloidal stability in water, the water soluble graft comprises polyethylene glycol.

6. The system of claim 3 wherein the functionalized amphiphilic graft copolymer comprises an anchoring group, wherein the anchoring group includes carboxylic acid or an alkoxysilane group.

7. The system of claim 1 wherein the remediation agent comprises one or more of:
a. one or more nanoparticles;
b. one or more nutrients; or
c. one or more microorganisms.

8. The system of claim 1 wherein the remediation site comprises a water/contaminant interface.

9. The system of claim 1 wherein the surrounding media comprises dissolved oxygen and/or water in a subsurface.

10. A targeted iron nanoparticle delivery system for remediation of groundwater contaminants at a water/contaminant interface, comprising:
a remediation agent having iron nanoparticles, the iron nanoparticles having neutralizing properties for remediating groundwater at the water/contaminant interface;
a functionalized amphiphilic graft copolymer adapted to entrap the remediation agent to protect from reactivity with surrounding media while in transport to and while at the water/contaminant interface to ensure the iron nanoparticles access and neutralize the contaminant.

11. The system of claim 10 wherein the functionalized amphiphilic graft copolymer comprises a polysiloxane polymer backbone to facilitate permeation of the contaminant onto a surface of the iron nanoparticle.

12. The system of claim 10 wherein the functionalized amphiphilic graft copolymer comprises a water soluble graft to facilitate dispersibility and colloidal stability in water.

13. The system of claim 12 wherein the water soluble graft comprises polyethylene glycol.

14. The system of claim 10 wherein the functionalized amphiphilic graft copolymer comprises an anchoring group, wherein the anchoring group comprises carboxylic acid or an alkoxysilane group.

15. The system of claim 10 wherein surrounding media comprises dissolved oxygen and/or water in a subsurface.

16. A method for targeted delivery of a remediation agent to a remediation site for remediation of a groundwater contaminant, the method comprising:
providing a copolymer transport;
entrapping the remediation agent within the copolymer transport;
transporting the remediation agent with the copolymer transport to the remediation site; and
keeping the remediation agent from reacting with surrounding media while in transport to and while at the remediation site to access and neutralize the groundwater contaminant.

17. The method of claim 16 wherein the remediation agent comprises an iron nanoparticle.

18. The method of claim 16 wherein the copolymer comprises a functionalized amphiphilic graft copolymer having a polysiloxane polymer backbone.

19. The method of claim 16 further comprising the step adding a water soluble graft of polyethylene glycol to the functionalized amphiphilic graft copolymer.

20. The method of claim 16 further comprising the step adding an anchoring group of carboxylic acid or an alkoxysilane group.

21. The method of claim 17 further comprising the step of protecting the iron nanoparticles from excessive oxidation by creating a barrier to the water and an affinity of the iron nanoparticles for the remediation site using inherent hydrophobicity of the polysiloxane polymer backbone to water.
